(19) United States
(12) Patent Application Publication
(10) Pub. No.: US 2025/0176885 A1
(43) Pub. Date: Jun. 5, 2025

(54) USER PERSONALITY PREDICTION METHOD BASED ON AUTOMATIC DATA COLLECTION, AND APPARATUS THEREFOR

(71) Applicant: Electronics and Telecommunications Research Institute, Daejeon (KR)

(72) Inventors: DAEWOOK KIM, Daejeon (KR); SEONG IL YANG, Daejeon (KR); SIHWAN JANG, Daejeon (KR)

(21) Appl. No.: 18/742,686

(22) Filed: Jun. 13, 2024

(30) Foreign Application Priority Data
Dec. 4, 2023 (KR) 10-2023-0173570

(51) Int. Cl. A61B 5/16

(52) U.S. Cl. CPC A61B 5/167

(57) ABSTRACT

Proposed is a method of predicting and analyzing a user's personality. The method is performed by a user personality prediction system, and includes obtaining data related to the user from a nearby device of the user, the data related to the user being collected and obtained for a particular period of time set by the user, predicting the user's personality using a machine learning model pre-trained on the basis of the data related to the user, and analyzing the predicted personality of the user to obtain analysis data.

### CROSS REFERENCE TO RELATED APPLICATION

The present application claims priority to Korean Patent Application No. 10-2023-0173570, filed Dec. 4, 2023, the entire contents of which is incorporated herein for all purposes by this reference.

### BACKGROUND OF THE INVENTION

### Field of the Invention

The present disclosure relates to a method of predicting and analyzing user personality in the real world by using data obtainable from various devices in both real and virtual worlds. More particularly, the present disclosure relates to a method of and an apparatus for predicting, inferring, and analyzing a user's personality using artificial intelligence learning techniques and data obtained through connection to various devices when the user uses a device in his or her daily life, although the user does not take time to answer questions in a survey.

### Description of the Related Art

As various digital content and services are created, customized services have been increased to improve service profitability. A technology previously used to provide customized services is content-based filtering. Content-based filtering is a method of breaking details of content into elements, identifying the elements of content that users like, and recommending content with similar elements. Recommendation performance of content-based filtering techniques depends on how the elements included in content are decomposed, and is low generally.

As a technology for providing customized services, there is collaborative filtering. Collaborative filtering uses many relationships between content and users to find users who consume similar content to a user who wants to be given a customized service, and then recommends content that the users have consumed but the user who wants to be given content has not consumed.

However, it is difficult to use these technologies when the amount of content is very large, such as games or metaverses, when multiple pieces of content must be consumed in sequence, or when the amount of content is very small.

In the meantime, recently, there have been various methodologies that represent a person's personality on the basis of psychological type theories, and the methodologies are widely known to the general public. For example, the Myers-Briggs Type Indicator (MBTI) is based on Carl Jung's theory of personality types. The MBTI test gives a person a numerical measure of four categories: introversion or extraversion, sensing or intuition, thinking or feeling, and judging or perceiving. Each of the four categories is divided into two types, making a total of 16 tendencies in the MBTI. Another theory is the Enneagram. The Enneagram originates from the Greek words “ennear” meaning “nine” and “grammos” meaning “figure”.

In addition, there are other tests including the Minnesota Multiphasic Personality Inventory (MMPI), the OCEAN test, also called the BIG5, and the DISC test developed by Willam Mouston Marston, a professor of psychology at Columbia University in the United States.

These personality tests are expensive and time-consuming. Mostly, answering pre-made questionnaires for personality tests takes about 60 minutes. There may be 300 questions or more. Therefore, it is difficult for the general public to widely use the tests, and there are also difficulties in utilizing services using the tests.

The foregoing is intended merely to aid in the understanding of the background of the present disclosure, and is not intended to mean that the present disclosure falls within the purview of the related art that is already known to those skilled in the art.

### SUMMARY OF THE INVENTION

The present disclosure is directed to providing a method of and apparatus for analyzing user disposition/personality from data obtained from various devices in the real world and log data of a user in virtual worlds and digital platforms.

The present disclosure provides a method of and an apparatus for predicting and analyzing a user's personality.

According to the present disclosure, there is provided a method of predicting and analyzing a user's personality, the method performed by a user personality prediction system and including: obtaining data related to the user from a nearby device of the user, the data related to the user being collected and obtained for a particular period of time set by the user; predicting the user's personality using a machine learning model pre-trained on the basis of the data related to the user; and analyzing the predicted personality of the user to obtain analysis data.

According to the present disclosure, there is provided an apparatus for predicting and analyzing a user's personality, the apparatus including a processor, wherein the processor is configured to obtain data related to the user from a nearby device of the user, the data related to the user being collected and obtained for a particular period of time set by the user, predict the user's personality using a machine learning model pre-trained on the basis of the data related to the user, and analyze the predicted personality of the user to obtain analysis data.

The nearby device of the user may be selected by the user.

The user's personality may be predicted on the basis of a personality test type selected by the user, and the personality test type may be at least one selected from a group of the MBTI, the Enneagram, and the Minnesota Multiphasic Personality Inventory.

The data related to the user may be related to at least one selected from a group of frequency of use of the nearby device, location information of the nearby device, and usage time of the nearby device.

The analysis data may include distribution of other users whose personality is the same as the predicted personality of the user.

When a plurality of the personality test types are selected by the user, the analysis data may include data related to a relationship between the user's personalities respectively based on the plurality of the personality test types.

The data related to the user may include data of a program executed by the user through the nearby device selected by the user.

The present disclosure provides a method of and an apparatus for analyzing user disposition/personality efficiently from data obtained from various devices in the real world and log data of a user in virtual worlds and digital platforms.

Effects that may be obtained from the present disclosure will not be limited to only the above described effects. In addition, other effects which are not described herein will become apparent to those skilled in the art from the following description.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a diagram illustrating a system for predicting and analyzing user personality according to an embodiment of the present disclosure;

FIG. 2 is a diagram illustrating a method of predicting and analyzing user personality according to an embodiment of the present disclosure;

FIG. 3 is a diagram illustrating a method of searching for nearby devices of a user and making connections according to an embodiment of the present disclosure;

FIG. 4 is a diagram illustrating a method of discovering the possibility of collecting digital data related to a user, and making connections according to an embodiment of the present disclosure;

FIG. 5 is a diagram illustrating a method of selecting a period of collection of digital data related to a user, selecting a personality test type, and agreeing to the collection of the digital data related to the user according to an embodiment of the present disclosure;

FIG. 6 is a diagram illustrating a system for predicting a user's personality according to an embodiment of the present disclosure; and

FIG. 7 is a diagram illustrating one of the apparatuses that performs a method of predicting and analyzing user personality, as one of the embodiments.

### DETAILED DESCRIPTION OF THE INVENTION

The terms used herein are general terms that are currently widely used, considering the functions in the present disclosure. However, the terms may have different meanings according to an intention of one of ordinary skill in the art, precedent cases, or the appearance of new technologies. In addition, some terms may be selected optionally by the applicant. In this case, the meaning of the selected terms will be described in the present disclosure. Therefore, the terms used herein should be construed in accordance with the actual meaning of the term and the content throughout the present disclosure, rather than just the name of the term.

In addition, a variety of modifications may be made to the present disclosure and there are various embodiments of the present disclosure. Particular embodiments are illustrated in the drawings and specifically described in the detailed description. However, the present disclosure is not limited thereto, and the exemplary embodiments can be construed as including all modifications, equivalents, or substitutes in a technical concept and a technical scope of the present disclosure. The similar reference numerals refer to the similar elements described in the drawings.

Terms used herein, “first”, “second”, “A”, “B”, etc. can be used to describe various elements, but the elements are not to be construed as being limited to the terms. The terms are only used to differentiate one element from other elements. For example, a “first” element may be named a “second” element without departing from the scope of the present disclosure, and similarly, a “second” element may also be named a “first” element. The term “and/or” includes a combination of a plurality of items or any one of a plurality of terms.

It should be understood that when an element is referred to as being “coupled” or “connected” to another element, it can be directly coupled or connected to the other element or intervening elements may be present therebetween. In contrast, it should be understood that when an element is referred to as being “directly coupled” or “directly connected” to another element, there are no intervening elements present.

Unless otherwise defined, all terms including technical and scientific terms used herein have the same meaning as commonly understood by those skilled in the art to which the present disclosure belongs. It will be further understood that terms used herein should be interpreted as having a meaning that is consistent with their meaning in the context of the present disclosure and the relevant art and will not be interpreted in an idealized or overly formal sense unless expressly so defined herein.

Hereinafter, exemplary embodiments according to the present disclosure will be described in detail with reference to the accompanying drawings.

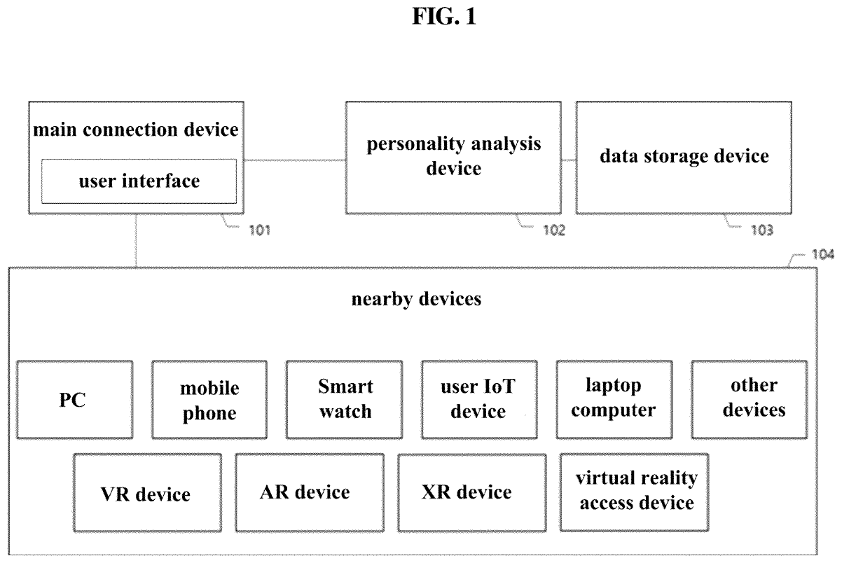

FIG. 1 is a diagram illustrating a system for predicting and analyzing user personality according to an embodiment of the present disclosure.

Specifically, FIG. 1 shows the configuration of apparatuses for automatically collecting at least one piece of data related to a user from one or more devices, and predicting and analyzing the user's personality on the basis of the collected data.

Referring to FIG. 1, the apparatuses for predicting and analyzing a user's personality may include a main connection device 101, a personality analysis device 102, a data storage device 103, and nearby devices 104. In addition, there may be an apparatus for predicting and analyzing a user's personality, the apparatus including a processor for controlling the main connection device 101, the personality analysis device 102, the data storage device 103, and the nearby devices 104. The processor may be a device, such as a processor, an application processor (AP), or a chip in which a program is embedded, for processing data and particular computations. For example, the processor may include a central processing unit (CPU), a graphics processing unit (GPU), or a neural processing unit (NPU).

The main connection device 101 may provide a user interface. The main connection device 101 may search for the nearby devices 104 selected by the user via the user interface and make connections to the nearby devices. The main connection device 101 may collect data obtained from the connected nearby devices 104 and forward the data to the personality analysis device 102 in real time. When necessary, the main connection device 101 may forward various types of data generated as the user uses the main connection device 101, to the personality analysis device 102 in order to predict the user's personality even if there is no nearby device 104. When the user wants, the main connection device 101 may receive a result of predicting the user's personality and analysis details from the personality analysis device 102 and output the result and the analysis details through the user interface.

The personality analysis device 102 may forward the various types of data provided from the main connection device 101 to the data storage device 103 as they are or after primary processing. In addition, when the personality analysis device 102 determines that sufficient data is collected (e.g., a particular threshold or more is reached), the personality analysis device 102 may load training data from the data storage device 103 and perform training. When the user requests information on the user's personality through the user interface of the main connection device 101, the personality analysis device 102 may use a machine learning model to infer and analyze the user's personality, and the personality analysis device 102 may forward an analysis result to the main connection device 101.

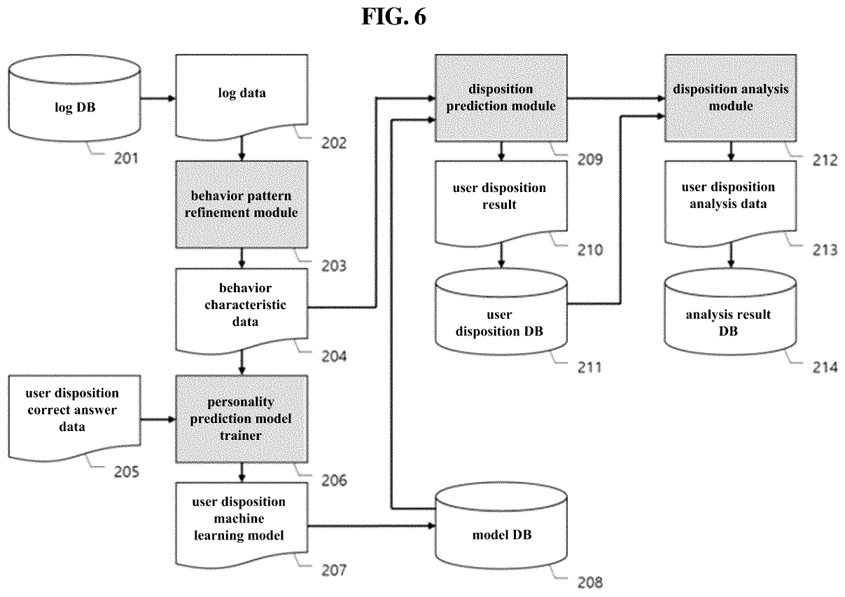

The data storage device 103 may store pieces of data obtained from the nearby devices 104 and intermediate results generated by the personality analysis device 102. The stored intermediate results may include a log DB 201, log data 202, behavior characteristic data 204, user disposition ground truth data 205, a trained model of user disposition 207, a model DB 208, a user disposition result 210, a user disposition DB 211, user disposition analysis data 213, and an analysis result DB 214, which will be described with reference to FIG. 6.

The nearby devices 104 may be devices that are required in terms of hardware and/or software to collect pieces of data required for personality prediction. For example, the nearby devices 104 may be a PC, a mobile phone, a smartwatch, a user IoT device, a laptop computer, a VR device, an AR device, an XR device, and a virtual reality access device.

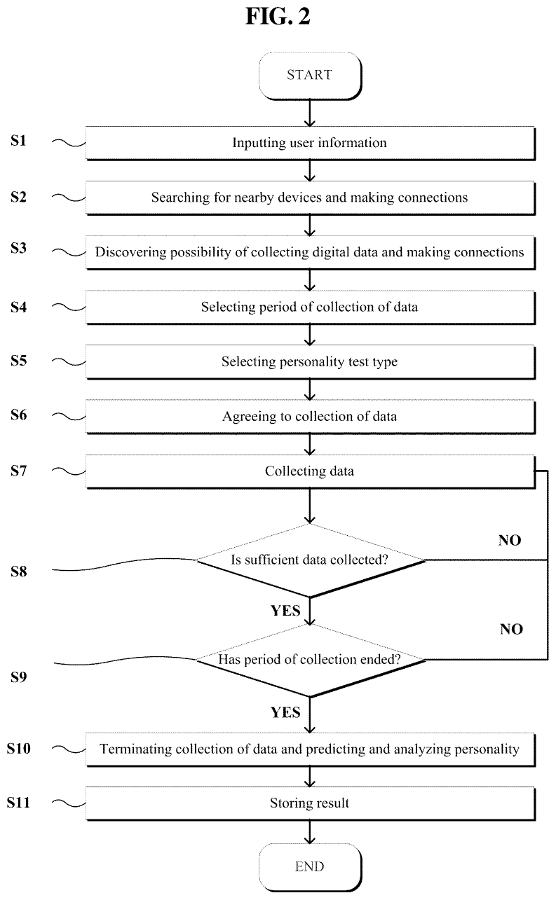

FIG. 2 is a diagram illustrating a method of predicting and analyzing user personality according to an embodiment of the present disclosure.

Referring to FIG. 2, a user may use the main connection device 101 to input the user's information in step S1. Herein, the user's information may include information, such as name, age, and sex. The main connection device 101 may search for the nearby devices 104 of the user and make connections to the nearby devices 104 of the user in step S2. Herein, the user may select one or more nearby devices to be connected. The main connection device 101 may discover the possibility of collecting digital data related to the user from the nearby devices 104 of the user and make connections in step S3. The user may select a period of collection of digital data related to the user in step S4. The user may select a personality test type that he or she wants in step S5. The user may select whether to agree to the collection of the digital data related to the user collected from the nearby devices 104 of the user in step S6. The main connection device 101 may collect the digital data related to the user from the nearby devices 104 of the user in step S7. Herein, step S7 may be performed when the user agrees to the collection of the digital data. The personality analysis device 102 may determine whether sufficient digital data related to the user is collected in step S8. The personality analysis device 102 may receive the digital data related to the user from the main connection device 101. When the personality analysis device 102 determines that sufficient digital data related to the user is collected, it may be determined whether the period of collection of the digital data related to the user has ended in step S9. Conversely, when the personality analysis device 102 determines that sufficient digital data related to the user is not collected, the digital data related to the user may be further collected. When the period of collection of the digital data related to the user has ended, the personality analysis device 102 may terminate the collection of the digital data related to the user. Conversely, when the period of collection of the digital data related to the user has not ended, the personality analysis device 102 may collect the digital data related to the user until the period of collection ends. The personality analysis device 102 may terminate the collection of the digital data related to the user, and predict and analyze the user's personality in step S10. The user personality analysis device 102 may store a result of predicting and analyzing the user's personality in step S11. Herein, the result for the user's personality may be stored in the data storage device 103, and may be provided to the user through the main connection device 101.

Steps S1 to S6 shown in FIG. 2 are part of a method of predicting and analyzing a user's personality and are merely an example, and the sequence (process) of steps S1 to S6 is not limited thereto.

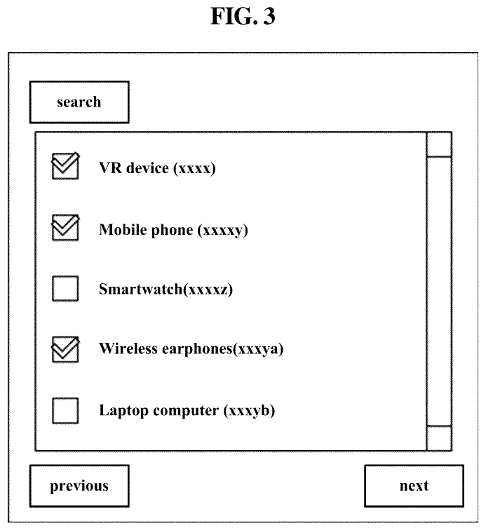

FIG. 3 is a diagram illustrating a method of searching for nearby devices of a user and making connections according to an embodiment of the present disclosure.

Specifically, FIG. 3 shows an example of performing step S2 (that is, the step of searching for the nearby devices of the user and making connections to the nearby devices of the user) described with reference to FIG. 2.

Referring to FIG. 3, through the user interface, the main connection device 101 may provide a result of searching for the nearby devices of the user. For example, the user may click a search button provided in the user interface, and the user interface may provide the found nearby devices of the user as a search result. Herein, the nearby devices of the user may include devices that are connectable in a wired/wireless manner. Referring to FIG. 3, the found nearby devices of the user may be a VR device, a mobile phone, a smartwatch, wireless earphones, and a laptop computer. The user may check the search result and select the nearby devices of the user for collecting the digital data related to the user. The user may select all or some of the found nearby devices of the user. For example, referring to FIG. 3, the user may select the VR device, the mobile phone, and the wireless earphones among the found nearby devices of the user, as devices for collecting the digital data related to the user. That is, the user may select the nearby devices of the user to be used to predict and analyze the user's personality.

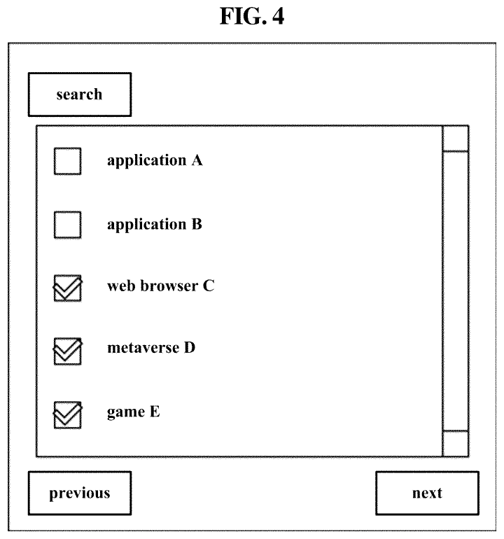

FIG. 4 is a diagram illustrating a method of discovering the possibility of collecting digital data related to a user, and making connections according to an embodiment of the present disclosure.

Specifically, FIG. 4 shows an example of performing step S3 (that is, the step of discovering the possibility of collecting digital data related to the user from the nearby devices 104 of the user, and making connections) described with reference to FIG. 2.

Referring to FIG. 4, the main connection device 101 may provide the user interface for selecting programs to be used for collecting the digital data related to the user. Through the user interface, the user may select programs to be used for collecting the digital data related to the user. Herein, the programs may be programs installed on the nearby devices of the user selected by the user described with reference to FIG. 3. For example, referring to FIG. 4, the programs installed on the nearby devices of the user selected by the user may include application A, application B, web browser C, metaverse D, and game E. The user may select all or some of the installed programs and request collection of the digital data related to the user about the selected programs. For example, the user may make selection so that the data related to the user while web browser C, metaverse D, and game E are running is used for predicting and analyzing the user's personality.

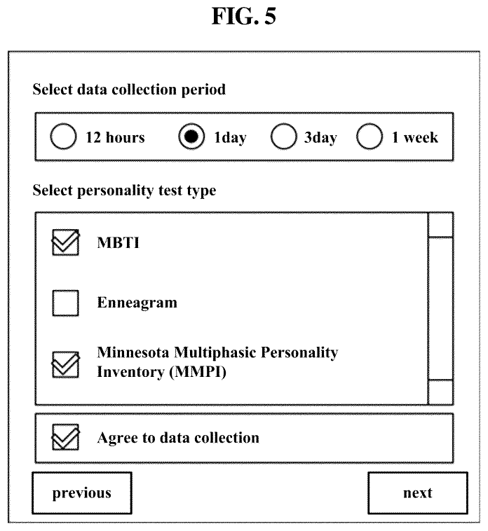

FIG. 5 is a diagram illustrating a method of selecting a period of collection of digital data related to a user, selecting a personality test type, and agreeing to the collection of the digital data related to the user according to an embodiment of the present disclosure.

Specifically, FIG. 5 shows an example of performing steps S4 to S6 (that is, the step of selecting a period of collection of data related to the user, selecting a personality test type, and agreeing to the collection of the digital data related to the user) described with reference to FIG. 2.

Referring to FIG. 5, the main connection device 101 may provide the user interface for the step of selecting a period of collection of data related to the user, selecting a personality test type, and agrecing to the collection of the digital data related to the user.

The user may select a period of collection of data related to the user. Examples of the period of collection may include minutes, hours, days, weeks, and months. For example, referring to FIG. 5, the user interface may provide the user with periods of collection including 12 hours, 1 day, 3 days, and 1 week, and the user may select any one of the provided periods of collection. During the selected period of collection, the digital data related to the user may be collected.

The user may select a personality test type that he or she wants. Referring to FIG. 5, examples of the personality test type may include the MBTI, the Enneagram, and the Minnesota Multiphasic Personality Inventory. The user interface may provide the user with the personality test types including the MBTI, the Enneagram, and the Minnesota Multiphasic Personality Inventory, and the user may select one or more of the personality test types. With the selected personality test type, the user's personality may be predicted and analyzed.

The user may select whether to agree to the collection of the digital data related to the user. Referring to FIG. 5, through the user interface, the user may select whether to agree to the collection of the digital data related to the user.

FIG. 6 is a diagram illustrating a system for predicting a user's personality according to an embodiment of the present disclosure.

A log DB 201 is present inside the data storage device 103 shown in FIG. 1, and may collect and manage pieces of log data 202. The above-described digital data related to the user may be divided into the log data 202, behavior characteristic data 204, and user disposition ground truth data 205.

The log data 202 may be any data obtainable from various devices. The various devices may include the user's nearby devices 104 shown in FIG. 1. For example, when one of the various devices is a PC, the log data 202 may include what programs are being used on the PC, how long the programs are being used, mouse and keyboard input, and screen images that are currently being output to a monitor. When one of the various devices is a mobile phone, the log data 202 may include what programs are being used on the mobile phone, how long the programs are being used, screen input, location information using a gyro sensor and GPS, ambient sound through a microphone, and images of surroundings through a camera. In addition, when an application is accessible internally or through an API, all data such as screen movement or state change within the application may be the log data 202.

A behavior pattern refinement module 203 may extract the log data 202 from the log DB 201 to refine the user's behavior pattern, and output the behavior characteristic data 204 corresponding to the refined behavior pattern.

The behavior characteristic data 204 may be data resulting from quantifying characteristic behaviors that may represent the user's personality. For example, when the behavior characteristic data 204 is about the user's behavior related to the mobile phone, the behavior characteristic data 204 may include how often the battery of the mobile phone is charged, the number of times that the screen of the mobile phone is turned on during a day (e.g., the average number of times during a day), the number of times that a particular application is executed during a day (e.g., the average number of times during a day), how long a particular application is executed, the number of times that a first web page is visited, the number of times that movement from the first web page to a second web page takes place, distribution of the user's movements based on GPS (e.g., distribution of the user's movements during a week), information of the user's location (e.g., whether the user is inside the house, and whether the user is within/outside a particular distance from the house) on a particular day (e.g., weekend), whether the user talks a lot with people around him or her, how often the user makes calls or uses messengers, and whether the user's voice tone is high or low when making calls.

The user disposition ground truth data 205 may be used when available to train a personality prediction model. The user disposition ground truth data 205 may be directly stored in the data storage device 103 by the user inputting information in advance through the user interface or by an administrator using data that he or she already has. In addition, when a trained model of user disposition exists in advance, the user disposition ground truth data 205 may be generated on the basis of a method such as semi-supervised learning.

A personality prediction model trainer 206 may perform machine learning using the behavior characteristic data 204 and the user disposition ground truth data 205 and output a trained model of user disposition 207. For machine learning, supervised learning may be often used. In addition, reinforcement learning may be used to improve prediction performance. When there is not a lot of behavior characteristic data 204, a tree-based method such as random forests may be more effective than a deep learning method.

The trained model of user disposition 207 may mean the output of training from the personality prediction model trainer 206. The trained model of user disposition 207 may be used by a disposition prediction module 209 to predict user disposition. When the trained model of user disposition 207 is the output based on a deep learning method, the trained model of user disposition 207 may be in the form of a large network structure composed of nodes and weighting values.

The model DB 208 may be a database (DB) in which the trained model of user disposition 207 is stored and managed. The performance of the trained model of user disposition 207 may be not perfect, so an administrator of the personality analysis device 102 may periodically refresh training. The existence of the model DB 208 may allow selection of a machine learning model with good performance among multiple machine learning models or analysis of whether synergy can be achieved (ensemble method) using several machine learning models simultaneously.

The disposition prediction module 209 may use the behavior characteristic data 204 and a machine learning model stored in the model DB 208 as input to predict the disposition of a user whose actual disposition is unknown and output a user disposition result 210.

The user disposition result 210 may be the output generated by operating the disposition prediction module 209 from log data 202 of a particular user. The user disposition result 210 may vary depending on a personality test type selected by the user.

A user disposition DB 211 may be a DB in which all user disposition results 210 are stored and managed.

A disposition analysis module 212 may receive the disposition prediction module 209 and the user disposition DB 211 as input to analyze the result of prediction and output user disposition analysis data 213. The disposition analysis module 212 may provide analysis of factors in the result of prediction of user disposition, and statistical information about the distribution of users having the same disposition. In addition, the disposition analysis module 212 may analyze a relationship between test results for several personality types, and a new form of personality clustering through behavior data of users.

The user disposition analysis data 213 may be data including various analysis results for user disposition.

An analysis result DB 214 may be a DB in which all pieces of user disposition analysis data 213 are stored and managed.

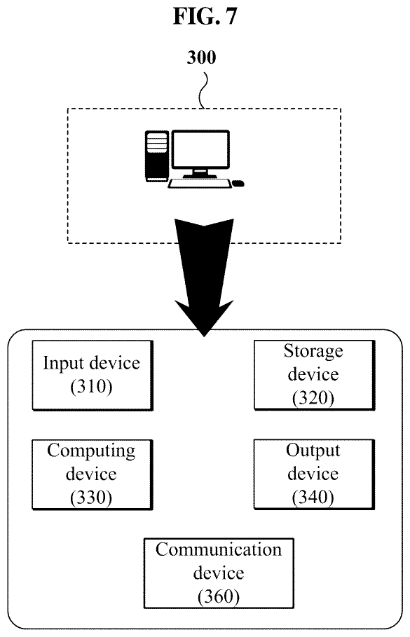

FIG. 7 is a diagram illustrating one of the apparatuses that performs a method of predicting and analyzing user personality, as one of the embodiments.

An analysis device 300 may be an apparatus that performs the above-described method of predicting and analyzing user personality. The analysis device 300 may include at least one input device 310, storage device 320, computing device 330, output device 340, and communication device 350. The input device 310 may receive data, information, or models that are required to perform the above-described method of predicting and analyzing user personality. The input device 310 may receive data related to the user. The input device 310 may receive a machine learning model. The input device 310 may receive training data required to train the machine learning model.

The input device 310 may include a device (a keyboard, a mouse, a touch screen, a joystick, a trackball, a touchpad, a scanner, or a webcam) for inputting particular commands or data. The input device 310 may also include an element for receiving data through a separate storage device (USB, CD, or hard disk). The input device 310 may receive data through a separate measurement device or a separate database. The input device 310 may receive data through the communication device 350 in a wired or wireless manner. The input device 310 may receive control signals for controlling the analysis device 300.

The storage device 320 may store data, information, or models that are required to perform the above-described method of predicting and analyzing user personality. The storage device 320 may store data information related to the user. The storage device 320 may store the machine learning model. The storage device 320 may store training data required to train the machine learning model. The storage device 320 may be a device that stores particular data, information, or models. The storage device 320 may store data, information, and models that are received through the input device 310. The storage device 320 may store instructions that enable the computing device 330 to perform the operations required for the method of predicting and analyzing user personality. The storage device 320 may store information generated during the computing process of the computing device 330. That is, the storage device 320 may include a memory. For example, the storage device may include a hard disk drive (HDD), a solid-state drive (SSD), ROM, RAM, a CD-ROM, a magnetic tape, or a floppy disk.

The computing device 330 may perform computations required to perform the above-described method of predicting and analyzing user personality. The computing device 330 may predict the user's personality using the machine learning model pre-trained on the basis of data related to the user. The computing device 330 may obtain analysis data by analyzing the predicted personality of the user.

The computing device 330 may be a device, such as a processor, an application processor (AP), or a chip in which a program is embedded, for processing data and particular computations. For example, the computing device 330 may include a central processing unit (CPU), a graphics processing unit (GPU), or a neural processing unit (NPU).

The computing device 330 may generate the control signals for controlling the analysis device 300. The computing device 330 may generate the control signals for controlling the input device 310, the storage device 320, the output device 340, and the communication device 350 included in the analysis device 300.

The output device 340 may be a device that outputs particular data, information, and models. The output device 340 may be a device that outputs particular data, information, and models to the outside of the analysis device 300. The output device 340 may output an interface required for a data processing process, input data, and analysis results. The output device 340 may include devices for outputting data through tactile, visual, audible, gustatory, and olfactory methods. The output device 340 may be implemented in physically various forms, such as a display, a speaker, a vibration motor, or a document output device. The output device 340 may output data, information, or models stored in the storage device 320. The output device 340 may output data, information, and models generated during the computing process of the computing device 330. The output device 340 may output results of computations by the computing device 330.

The communication device 350 may receive information required to perform the above-described method of predicting and analyzing user personality. The communication device 350 may receive a model required to perform the above-described method of predicting and analyzing user personality. The communication device 350 may transmit and receive data information related to the user. The communication device 350 may transmit and receive the machine learning model. The communication device 350 may receive control signals required to control the analysis device 300. The communication device 350 may transmit analysis results obtained by the analysis device 300. The communication device 350 may mean an element for receiving and transmitting particular data, information, and models over a wired or wireless network. The communication device 350 may perform network communication, such as Wireless Fidelity (Wi-Fi), Wi-Fi Direct, Bluetooth, ultra-wide band (UWB), near-field communication (NFC), Universal Serial Bus (USB), High-Definition Multimedia Interface (HDMI), or a local area network (LAN).

According to a method of predicting and analyzing a user's personality described herein, users can recognize their personality without having to take the time to answer complex and time-consuming personality test questions. In addition, when content providers want to provide customized services, the content providers can make analogy through data inference even if the content providers do collect disposition/personality test result information individually from users, thereby providing services according to user disposition.

The methods according to the present disclosure may be realized in a program instruction format that may be executed by using diverse computing means, so as to be recorded in a computer-readable medium. The computer-readable medium may include program instructions, data files, and data structures separately or in combinations. The program instructions recorded on the computer-readable medium may be specially designed and configured for the present disclosure or may be well-known to and be usable by those skilled in the art of computer software.

Examples of the computer-readable medium may include hardware devices, such as ROM, RAM, and flash memory, specially configured for storing and executing the program instructions. Examples of the program instructions include not only a mechanical language code created by a compiler but also a high-level language code that may be implemented by a computer using an interpreter. The hardware devices may be configured to operate as one or more software modules to perform the operation of the exemplary embodiment, and vice versa.

In addition, a part or whole of the configurations or functions of the above-described method or apparatus may be implemented in a combined manner or separately. The description of the present disclosure is illustrative. It will be understood by those skilled in the art that the present disclosure can be embodied in other specific forms without changing the technical idea or essential characteristics of the present disclosure. Therefore, it should be understood that the embodiments described above are illustrative in all aspects as and not restrictive. For example, each element described as unitary may also be implemented in a distributed manner. Similarly, elements described as distributed may also be implemented in a combined form.

Although the present disclosure has been described with reference to the exemplary embodiments, those skilled in the art will appreciate that various modifications and variations can be made in the present disclosure without departing from the spirit or scope of the disclosure described in the appended claims.

What is claimed is:

1. A method of predicting and analyzing a user's personality, the method performed by a user personality prediction system and comprising:
obtaining data related to the user from a nearby device of the user, the data related to the user being collected and obtained for a particular period of time set by the user;
predicting the user's personality using a machine learning model pre-trained on the basis of the data related to the user, and
analyzing the predicted personality of the user to obtain analysis data.

2. The method of claim 1, wherein the nearby device of the user is selected by the user.

3. The method of claim 1, wherein the user's personality is predicted on the basis of a personality test type selected by the user, and
the personality test type is at least one selected from a group of the MBTI, the Enneagram, and the Minnesota Multiphasic Personality Inventory.

4. The method of claim 1, wherein the data related to the user is related to at least one selected from a group of frequency of use of the nearby device, location information of the nearby device, and usage time of the nearby device.

5. The method of claim 1, wherein the analysis data includes distribution of other users whose personality is the same as the predicted personality of the user.

6. The method of claim 3, wherein when a plurality of the personality test types are selected by the user, the analysis data includes data related to a relationship between the user's personalities respectively based on the plurality of the personality test types.

7. The method of claim 1, wherein the data related to the user includes data of a program executed by the user through the nearby device selected by the user.

8. An apparatus for predicting and analyzing a user's personality, the apparatus comprising a processor,
wherein the processor is configured to obtain data related to the user from a nearby device of the user, the data related to the user being collected and obtained for a particular period of time set by the user,
predict the user's personality using a machine learning model pre-trained on the basis of the data related to the user, and
analyze the predicted personality of the user to obtain analysis data.

9. The apparatus of claim 8, wherein the nearby device of the user is selected by the user.

10. The apparatus of claim 8, wherein the user's personality is predicted on the basis of a personality test type selected by the user, and
the personality test type is at least one selected from a group of the MBTI, the Enneagram, and the Minnesota Multiphasic Personality Inventory.

11. The apparatus of claim 8, wherein the data related to the user is related to at least one selected from a group of frequency of use of the nearby device, location information of the nearby device, and usage time of the nearby device.

12. The apparatus of claim 8, wherein the analysis data includes distribution of other users whose personality is the same as the predicted personality of the user.

13. The apparatus of claim 10, wherein when a plurality of the personality test types are selected by the user, the analysis data includes data related to a relationship between the user's personalities respectively based on the plurality of the personality test types.

14. The apparatus of claim 8, wherein the data related to the user includes data of a program executed by the user through the nearby device selected by the user.
